(19) United States
(12) Patent Application Publication
(10) Pub. No.: US 2020/0171048 A1
(43) Pub. Date: Jun. 4, 2020

(54) PREVENTION OF BOAR TAINT AND AGGRESSION

(71) Applicant: lnsigna lnc., Champaign, IL (US)

(72) Inventor: CheMyong Ko, Champaign, IL (US)

(21) Appl. No.: 16/699,307

(22) Filed: Nov. 29, 2019

Related U.S. Application Data
(63) Continuation-in-part of application No. 16/526,874, filed on Jul. 30, 2019.
(60) Provisional application No. 62/772,878, filed on Nov. 29, 2018.

(51) Int. Cl. A61K 31/565; A01K 21/00; A61K 9/00

(52) U.S. Cl. CPC A61K 31/565; A01K 2227/108; A61K 9/0019; A01K 21/00

(57) ABSTRACT

Methods, compositions and systems are presented to deliver estradiol benzoate to prevent and/or inhibit boar taint and/or male aggression as compared to animal that has not been administered estradiol.

### CROSS-REFERENCE TO RELATED APPLICATIONS

This application claims the benefit of priority under 35 U.S.C. § 119(e) to U.S. Provisional Application No. 62/772,878, filed on Nov. 29, 2018, the disclosure of which is incorporated herein by reference in its entirety. This application is also a continuation-in-part of U.S. application Ser. No. 16/526,874 and claims the benefit of priority to U.S. application Ser. No. 16/526,874 filed Jul. 30, 2019, the disclosure of which is incorporated herein by reference in its entirety.

### BACKGROUND

In food animals such as bovines and swine, castration is performed on males as a means of decreasing testosterone production. The presence of testosterone in a food animal causes unpleasant, pungent odors when its meat is processed and cooked (taint), particularly in meat from swine. Castration can also result in a higher grade of meat, i.e., meat that is leaner and has a higher content of intramuscular fat, also known as marbling, particularly in bovine meat. Furthermore, castration reduces aggression, particularly by the male animals, decreasing potential physical hazards to other livestock or to human handlers.

Currently, castration in the males of both companion and food animals is achieved primarily by surgical methods: physical removal of testes in males. However, multiple issues exist with the use of physical castration methods in food animals such as boars and bulls, primarily because the procedures are generally performed without anesthesia. Such castration methods cause severe pain to the animal subject, raise concerns for humane treatment of animals and impact the morale of the performer of the castration.

In some male food animals such as bulls, castration is achieved by non-surgical methods, such as the use of an elastomeric ring to block or otherwise damage the blood supply to the testicles, leading to the demise of testicular tissue. Typically, however, both surgical and non-surgical castration of livestock are performed without anesthesia or other pain management, which inevitably causes great discomfort and pain in the animals.

### SUMMARY

The use of estradiol benzoate (EB) as a novel method of preventing, reducing or inhibiting male meat taint and aggression is described herein.

One embodiment provides a method to inhibit boar taint and/or aggression comprising administering to said pig an effective amount of estradiol, estradiol benzoate, estradiol dipropionate, estradiol valerate, estradiol cypionate or a combination thereof so as to prevent or inhibit boar taint and/or aggression. In one embodiment, estradiol, estradiol benzoate, estradiol dipropionate, estradiol valerate, estradiol cypionate, or a combination thereof is administered in a composition comprising a carrier, such as a physiologically acceptable oil. In another embodiment, the estradiol, estradiol benzoate, estradiol dipropionate, estradiol valerate, estradiol cypionate, or a combination thereof is formulated in a slow release formulation. In one embodiment, the administration is a single dose, one time, administration. In another embodiment, the estradiol, estradiol benzoate, estradiol dipropionate, estradiol valerate, estradiol cypionate, or a combination thereof is administered intraperitoneally, intramuscularly, or subcutaneously. In one embodiment, the estradiol, estradiol benzoate, estradiol dipropionate, estradiol valerate, estradiol cypionate, or a combination thereof is administered prior to puberty, such as within 1-7 days after birth, such as 2 day after birth (or on the date of birth). In one embodiment, between about 0.3 mg to about 11.0 mg estradiol, estradiol benzoate, estradiol dipropionate, estradiol valerate, estradiol cypionate, or a combination thereof is administered to the male piglet, including about 3 mg or about 9.0 mg. In one embodiment only one (single) administration is given per animal. In another embodiment, the estradiol, estradiol benzoate, estradiol dipropionate, estradiol valerate, estradiol cypionate, or a combination thereof can further be combined with vaccines, iron and/or other agents of benefit to the animal.

This summary is intended to provide an overview of the subject matter of the present disclosure. It is not intended to provide an exclusive or exhaustive explanation of the invention. The detailed description is included to provide further information about the present disclosure.

### Abbreviations

EB Estradiol benzoate

E2 Estradiol

AVPV Anteroventral Periventricular nucleus

ARC Arcuate nucleus

POA Preoptic area

KISS1 Kisspeptin protein

Kiss1 Kisspeptin gene or mRNA.

LHβ Luteinizing Hormone subunit beta

GH Growth Hormone

CASA Computer assisted semen analysis

### BRIEF DESCRIPTION OF THE DRAWINGS

The drawings illustrate generally, by way of example but not by way of limitation, various embodiments discussed in the present document. In the drawings, which are not necessarily drawn to scale, like numerals may describe similar components in different views. Like numerals having different letter suffixes may represent different instances of similar components.

FIGS. 1A-D demonstrate E2 profile (EB300; also designated as iNeuter herein) and the impact on serum testosterone and fertility in male rats.

FIGS. 2A-B provide the effect of EB300 on testis and seminal vesicle development in male rats.

FIG. 3 provide patterns of body weight growth (Left) and testicular volume change (Middle) during the period of 4 weeks of ages to 12 weeks of ages. Also presented are the serum testosterone concentration measured at the age of 10 weeks (Right).

FIG. 4 is a cross-sectional side view of an example injector device for use in a chemical-sterilant delivery system (for example, subcutaneous delivery to a piglet).

### DETAILED DESCRIPTION

The following detailed description includes references to the accompanying drawings, which form a part of the detailed description. The drawings show, by way of illustration, specific embodiments in which the invention may be practiced. These embodiments, which may also be referred to herein as “examples,” are described in enough detail to enable those skilled in the art to practice the invention. The example embodiments may be combined, other embodiments may be used, or structural, and logical changes may be made without departing from the scope of the present invention. While the disclosed subject matter will be described in conjunction with the enumerated claims, it will be understood that the exemplified subject matter is not intended to limit the claims to the disclosed subject matter. The following detailed description is, therefore, not to be taken in a limiting sense, and the scope of the present invention is defined by the appended claims and their equivalents.

### Definitions

In this document, the terms “a” or “an” are used to include one or more than one and the term; “or” is used to refer to a nonexclusive “or” unless otherwise indicated. In addition, the phraseology or terminology employed herein and not otherwise defined is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

References in the specification to “one embodiment,” “an embodiment,” “an example embodiment,” etc., indicate that the embodiment described can include a particular feature, structure; or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure; or characteristic in connection with other embodiments whether or not explicitly described.

Values expressed in a range format should be interpreted in a flexible manner to include not only the numerical values explicitly recited as the limits of the range but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt. % to about 5 wt. %, but also the individual concentrations (e.g., 1%, 2%, 3%, and 4%) and the sub-ranges (e.g., 0.1% to 0.5%, 1.1% to 2.2%, and 3.3% to 4.4%) within the indicated range.

The term “about” as used herein can allow for a degree of variability in a value or range—for example, within 10%, within 5%, within 1%, within 0.5%, within 0.1%, within 0.05%, within 0.01%, within 0.005%, or within 0.001% of a stated value or of a stated limit of a range—and includes the exact stated value or range.

In addition to boar, bovine (e.g, bulls) can be subject to the compositions and methods of the invention as well. As used here, swine, boar and pig are interchangeable and includes any animal in those families.

As used herein, an “effective amount” means an amount sufficient to induce infertility (i.e., make the animal sterile). An effective amount can be administered in one or more administration. In some embodiments, an effective amount of EB can be achieved in conjunction with another drug, compound, or pharmaceutical composition. In other embodiments, an effective amount of EB may be achieved in isolation from the use of another drug, compound, or pharmaceutical composition.

The terms “carrier,” “pharmaceutically acceptable carrier,” or “physiologically acceptable carrier” as used herein refer to one or more formulation materials suitable for accomplishing or enhancing the delivery of EB as a composition (i.e., pharmaceutical composition).

### Compositions and Methods

Described herein are compositions that comprise estradiol benzoate (EB) and methods to use estradiol benzoate to prevent or inhibit male meat taint and/or aggression in male animals. Other compounds that may be used in the methods described include, but are not limited to, estradiol, estradiol dipropionate, estradiol valerate, estradiol cypionate (such that any form of chemical compound that releases estradiol or functionally acts like estradiol in vivo may be used). EB has the following chemical structure:

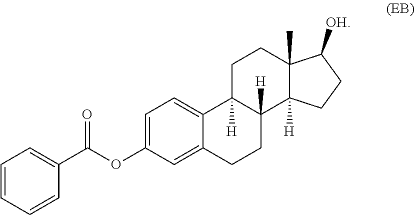

### Concentration/Amount of EB

Taint/aggression inhibiting compositions comprise an infertility-inducing amount of EB. An effective amount of EB to induce infertility can depend, for example, upon the animal species, the route of administration, the age of the animal, and its size (body weight). Accordingly, the skilled artisan may titer the dosage and modify the route of administration of EB to obtain the optimal effect for a particular animal. A typical dosage of EB may range from about 0.01 mg/kg to up to about 100 mg/kg or more. In other embodiments, the dosage of EB may range from 0.1 mg/kg up to about 100 mg/kg; or 1 mg/kg up to about 100 mg/kg; or 5 mg/kg up to about 100 mg/kg. For example, in rats and hamsters, 300, 100 or 30 μg can be administered to induce infertility. In larger animals, a similar dose would be effective. In pigs the dose can range from about 0.3 mg to about 11 mg per animal, including about 0.3, about 0.4, about 0.5, about 0.6, about 0.7, about 0.8, about 0.9, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, or 11 mg per animal.

### Timing of Administration

Compositions comprising EB to reduced taint and/or aggression are administered prior to puberty (prior to reaching sexual maturity/capable of reproduction). The compositions can be administered days, weeks, or even months after birth, as long as the compositions are administered before the animal reaches puberty. Administration of EB effectively inhibits/blocks maturation of sex organs/gonads in males.

### Route of Administration

The route of administration of the composition provided herein is in accordance with known methods, e.g., injection (intraperitoneal, intramuscular, subcutaneous) and nasal (inhalation). In one embodiment, EB is administered for inhibition of taint and/or aggression of an animal in a single, one-time dose. In other embodiments, multiple administrations of EB can be carried out to produce sterilization or inhibit taint and/or aggression.

### Compositions

In one embodiment, EB compositions for injectable administration can be in the form of oleaginous suspensions, including oil, such as vegetable oil (e.g., corn oil), cottonseed oil, peanut oil, and/or sesame oil. Other carriers or fillers can be used instead of, or in addition to, oil. Carriers/fillers can include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. These suspensions can be formulated according to methods available to the art for dispersing and suspending ingredients.

in another embodiment, the composition described above can be encapsulated for administration. In one embodiment, a capsule can be formed from silicone tubing with plugs at each end to contain a mixture of, for example, EB and oil. The capsules can be placed, such as by injection (further described below), in the body of the subject. The EB compositions described herein can be formulated for immediate release or in a time release formulation (e.g., slow release). For example, EB can be prepared with carriers that protect EB against rapid release, such as a controlled release formulation.

Many methods for the preparation of controlled/slow release formulations are known to those skilled in the art. For example, techniques for formulating a variety of sustained- or controlled-delivery means, such as liposome carriers, polymers (e.g., ethylene vinyl acetate, polyanhydrides, silicone, polyglycolic acid, collagen, polyorthoesters, polylactic acid and polylactic, polyglycolic copolymers (PLG)), microparticles, nanoparticles (such as nanospheres, including biodegradable nanospheres or porous beads, and depot injections) are also known to those skilled in the art. For example, see PCT/US93/00829, which describes controlled release of porous polymeric microparticles for the delivery of pharmaceutical compositions. Additional examples of sustained-release preparations include semipermeable polymer matrices in the form of shaped articles, e.g., films or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556, 1983), poly (2-hydroxyethyl-methacrylate) (Langer et al., J. Biomed. Mater. Res., 15; 167-277, 1981; Langer et al., Chem. Tech. 12:98-105,1982), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. USA, 82:3688-3692, 1985; EP 36,676; EP 88,046; EP 143,949.

In some embodiments, the composition has various release rates (e.g., controlled release or immediate release). Immediate release refers to the release of EB immediately upon administration. In another embodiment, immediate release occurs when there is dissolution of EB within 1-20 minutes after administration. Dissolution can be of all or less than all (e.g., about 70%, about 75%, about 80%, about 85%, about 90%, about 91%, about 92%, about 93%, about 94%, about 95%, about 96%, about 97%, about 98%, about 99%, about 99.5%, 99.9%, or 99.99%) of EB. In another embodiment, immediate release results in complete or less than complete dissolution within about one hour following administration.

Controlled-release, slow release, or sustained-release refer to the release of an active ingredient, such as EB, from a composition or dosage form in which the active ingredient is released over an extended period of time. In one embodiment, controlled-release results in dissolution of EB within 20-180 minutes after administration. In another embodiment, controlled-release results in dissolution over several hours to a few days or weeks. In another embodiment, EB is released over a period of a few weeks, including about 15 to 20 days.

In another embodiment, the composition is formulated for inhalation; for example, EB can be formulated as a dry powder. Inhalation solutions may also be formulated with a propellant for aerosol delivery. In another embodiment, inhalation solutions may be nebulized.

One embodiment provides kits for producing a single-dose administration unit. The kits may contain single and multi-chambered pre-filled syringes containing EB and instructions for use (sterilizing an animal).

### Delivery System Embodiment

The present disclosure also describes systems and methods for delivering a chemical composition (also referred to as “a chemical delivery system”) into an animal. In one embodiment, the chemical is EB.

FIG. 4 shows a conceptual view of an example chemical delivery system 10 (referred to hereinafter as “the system 10”). This system 10 example includes an injector device 12 (also referred to hereinafter as “the injector 12”) that is configured to deliver a chemical containment body 14 (e.g., capsule) onto or into an animal 2 (shown as a piglet 2 in FIG. 4, which could be a male or female piglet). Those having skill in the art will appreciate that the animal 2 could be male or female and could be another type of livestock, such as a piglet or lamb, or could be a companion animal, such as a dog or cat. The chemical containment body 14 includes a specified dose of the chemical (e.g., EB) and is configured to release the chemical into or onto the animal 2 after the chemical containment body 14 has been delivered to the animal 2 by the injector 12. In an example, the chemical containment body 14 is generally in the shape of a capsule, e.g., a cylindrical or substantially cylindrical body, so that it can pass through the injector 12 and into or onto the animal 2, as described in more detail below. For this reason, the chemical containment body 14 will also be referred to as “the delivery capsule 14,” or simply “the capsule 14.” Those having skill in the art will appreciate that the delivery body 14 need not have the cylindrical or substantially cylindrical shape that might ordinarily be thought of as a “capsule.”

In an example, the injector 12 comprises a main body 16, a force member 20 (e.g., plunger), and, in some examples, an insertion structure 18 and/or one or more grip members 22. In the example shown in FIG. 4, the injector 12 configuration is similar to a medical syringe such that in some examples the injector 12 will be referred to as a “syringe 12,” the main body 16 will be referred to as the “barrel 16,” and the force member 20 will be referred to as the “piston 20.” In an example, the main body 16 at least partially encloses a chamber 24 in which a portion of the force member 20 sits. In an example, the force member 20 can slide along the chamber 24 to change the internal volume experienced by the gas or fluid in the chamber 24 to change the pressure of the gas or fluid in the chamber 24, and thus, to change the driving force exerted by the gas or fluid in the chamber 24.

In an example, the insertion structure 18 (optional) comprises the structure though which the delivery capsule 14 will pass to provide for placement of the delivery capsule 14 at a specified location in or on the animal 2. In an example, the insertion structure 18 comprises a narrow structure with a small cross-sectional area allowing the insertion structure 18 to be used to pierce one or more epidermal layers of the animal 2 so that the delivery capsule 14 can be injected into it. For this reason, the insertion structure 18 may also be referred to hereinafter as “the hypodermic needle 18,” or simply “the needle 18.” For example, the needle 18 can include a cannula 26 that extends from a proximal end coupled to the barrel 16 and a distal end configured for insertion into the animal 2. The distal end of the cannula 26 can include a relatively sharp pointed tip that can pierce the one or more epidermal layers of the target animal 2. A lumen 30 passes through the cannula 26 from the proximal end to the distal end. In an example, the delivery capsule 14 can be positioned in the lumen 30 where the delivery capsule 14 can be driven out of the cannula 26. In an example, the lumen 30 is in fluid communication with the syringe chamber 24 so that the force member 20, when moved forward, can drive the delivery capsule 14 through the lumen 30 and out the distal tip with sufficient force to be injected into the target animal 2.

The force member 20 includes a movable structure that, when moved from a first position to a second position, can cause the delivery capsule 14 to be moved out of the injector 12 so that the delivery capsule 14 can be delivered into or onto the animal 2. In the example where the injector 12 is a syringe-like device, the main body 16 can be shaped in a cylindrical tube-like configuration, such that the main body 16 may also be referred to herein as “the barrel 16.” The force member 20 can comprise a piston that fits tightly within the cylindrical barrel 16 (e.g., with a structure having substantially the same cross-sectional shape and size as a cross section of the barrel 16) so that when the force member 20 is moved axially along the barrel 16, it compresses gas located within the chamber 24 (e.g., air), and the increased pressure acts as a driving force to drive the delivery capsule 14 through the insertion structure 18. In another example, the force member 20 includes a pushing structure 28 that is coupled to or that is part of the piston 20, wherein the pushing structure 28 engages the delivery capsule 14 and pushes the delivery capsule 14 out from the injector 12. For example, if the injector 12 includes an insertion structure 18, then the pushing structure 28 pushes the delivery capsule 14 through the lumen 30 of the insertion structure and out of the distal tip of the cannula 26 for insertion into the target animal. In an example, the one or more grip members 22 are disposed on the main body 16 of the injector 12 and allow a user to hold and stabilize the injector 12 while actuating the force member 20 to drive the delivery capsule 14 into the target animal 2.

In an example, the delivery capsule 14 is sized and shaped so that it fits within the lumen 30 in the insertion structure 18. In an example, the delivery capsule 14 is sized and shaped for intramuscular, intraperitoneal, or subcutaneous injection into the animal 2.

The system 10 shown in FIG. 4 has been found to be effective in providing for chemical-based sterilization of mammals and to inhibit taint and/or aggression in male animals. For example, in experiments (described in more detail in the EXAMPLES below and as mentioned above) where the sterilant delivery capsule 14 containing a sterilant composition 34 that includes estradiol-benzoate was injected subcutaneously to premature male rats, the sterilant composition 34 was found to be effective in inhibiting development of the neuron Kisspeptin in the hypothalamus of the brain, the testes (e.g., the site of testosterone and sperm production), and the seminal vesicle and prostate (e.g., accessory male reproductive glands that produce semen components). The male rats that were injected with the delivery capsule 14 containing EB were prevented from undergoing puberty and became permanently sterile. Specifically, the male rats' testicles did not produce testosterone or sperm. When the sterilant delivery capsule 14 with the estradiol-benzoate sterilant composition 34 was injected into premature female rats, the estradiol-benzoate was found to inhibit the females' reproductive systems, preventing the females from undergoing menstrual cycling and from producing ova.

The subject rats also did not have had any obvious negative health effects as a result of being injected with the estradiol-benzoate via the sterilant delivery capsule 14. An examination of the animals' immune systems, liver, and kidney functions found no clinical pathology in either the male or female subject rats. Moreover, after testing of more than 200 animals with the estradiol-benzoate-containing delivery capsule 14, no subject animal died or exhibited sickness during a six-month testing period.

The development and function of the reproductive system is consistent throughout all mammalian species. All mammals use the same reproductive hormones with minimal variations among species. For example, reproductive hormones such as estrogen, progesterone, Kisspeptin, luteinizing hormone (LH), and follicle stimulating hormone (FSH) extracted from one species have the same or substantially similar biological effect in all or nearly all other mammalian species. Because of the high level of conservation, estradiol-benzoate sterilant composition 34 (and other estradiol type compositions) will be effective in sterilizing all mammals and avians.

### EXAMPLE

Various embodiments can be better understood by reference to the following Example, which is offered by way of illustration. The disclosure is not limited to the Examples given herein.

### Example 1

### Prevention or Inhibition of Boar Taint and Animal Aggression

### Introduction

Surgical castration is a long-standing practice performed on farms that raise hogs for meat. In 2018, the US produced over 133 million pigs, or over 10% of the global supply (1). Generally, pig castration is performed within 10 days after birth without anesthesia (2, 3). Assuming 50% of pigs taken to market are males, almost 67 million pig castrations would have been performed in 2018 in the US alone. For this application, ‘surgical castration’ of pigs refers to nonsterile, physical extractions of male piglet gonads in a farm setting, without the use of anesthesia or analgesics and without sutures to close the incision. This procedure is considered necessary to improve the quality of meat by preventing or inhibiting boar-taint or unpleasant odor and taste that occurs when the boar meat is cooked. Castration also reduces aggression and thus improves the handling and management of boars as they grow.

The need to prevent the risk of boar taint for marketability of the meat is clear; however, the currently accepted method of castration raises issues for the industry and its stakeholders: 1) Animal welfare concerns associated with the pain induced from physically removing testicles without anesthesia; 2) Such undesirable process on the farm that negatively affects employee morale and the risk of employee injury, and 3) Alternatives to castration are cost or process prohibitive, making them financially unattractive to farm/business owners.

Animal Welfare: In pigs, castration is performed soon after birth because it is easier for farm staff to handle the animals when they are small and it has been generally assumed that at this age the animals would experience less pain and would recover more rapidly, although a recent study does not support this belief (4). The use of anesthesia on the farm is not only costly, but also impractical to perform on pigs in a farm environment because it requires too much time, monitoring, and robust process implementation. Further, the physical process of cutting scrotum and removing of the testicles causes severe pain and trauma even in the piglet, thus receiving increased attention from animal welfare advocates and government agencies in the US and abroad (5-7). In addition to the EU's attempt to ban surgical castration, some EU countries have moved forward with their own regulations requiring anesthesia or analgesics when performing surgical castration in order to continue the practice humanely (8-10). In both the US and EU, castrations after 14 days are mandated to be performed with analgesia and/or anesthesia by the respective governing agencies.

Employee morale: The daily castration of piglets, although routine, is a highly undesirable procedure on the farm. Cutting and pulling the testicles in fully awake pigs causes significant pain and stress (2, 4, 11-13) and negatively affects employee morale. Employees also suffer injuries from scalpel use on struggling piglets and repetitive motion problems that are common for workers in the farrowing barns (14).

Alternatives are cost prohibitive: Currently, there is only one FDA approved alternative to surgical castration. Zoetis's immuno-contraceptive Improvest® is an FDA approved immunocastration drug that targets gonadotropin-releasing hormone (GnRH) in the hypothalamus and inhibits pituitary stimulation of the testis to produce androgens. However, this drug requires two injections, as the first injection given at 10 weeks of age only gives a temporary reduction in testicular androgen synthesis and the risk of boar taint is high (15-17). The second injection given at 14 weeks after birth temporarily extends the inhibition, making this method more laborious and dangerous for the farm staff to administer, and costlier than single-injection solution. Industry contacts have indicated that this added requirement of a second injection, and the increased costs, coupled with staff concerns with handling the boars that retain aggressive behaviors until after the second injection, are the primary reasons the drug has not been adopted by US farmers.

It is common knowledge that castration of male pigs by a nonsterile, surgical procedure has been used on swine farms for centuries (20). As mentioned previously, castration is performed primarily to prevent the boar taint produced when the meat is cooked and also to reduce boar aggression (21). The reason why castration prevents boar taint is because it removes the testicular Leydig cells, the major source of circulating androgens, testosterone (T) and androstenone (Andro) (22, 23), which are found with nearly equal circulating concentrations in the boar (24). T stimulates boar aggression while Andro, which is uniquely found in high concentrations in male swine, contributes to boar taint as it builds up in adipose tissues, along with skatole (3-methyl-indole) produced in the gut (21-23). Skatole is removed metabolically in the liver (25), but Andro inhibits skatole metabolism and removal (25). Therefore, eliminating androgens produced by testes has led to the routine practice of castration in pigs to prevent the pungent odors that reduce the market acceptance of pork from non-castrated males (26, 27). Despite its routine use on the pig farms, there is a growing need to find an alternative to surgical castration. The literature clearly demonstrates that numerous efforts have been made to intervene the multiple steps in this hormonal cascade as a way to reduce androgen production, including the following: a GnRH agonist that decreases T and Andro (28); immunization against Andro (22) (29); immunization against LH (30); neonatal injections of T (23); and various types of immunizations against GnRH (15, 31-34). However, each of these attempts has run into significant problems along the way, including the most recent use of improvest®, which was pointed out above.

In the boar, pituitary hormone LH stimulates Leydig cells of the testis to produce androgens, including T and Andro, which contribute to the problems that have necessitated castration in the maturing pigs. T is also required for early development of elongating spermatids, terminal differentiation, and release of sperm (37). Androgen production is stimulated by LH. Upon stimulation by LH, Leydig cells of the testis produces androgens. Testosterone then travel to the hypothalamus, where this hormone inhibits KISS1 secretion from KISS1 neuron leading to the inhibition of GnRH secretion from GnRH neuron followed by decreased LH secretion from the pituitary. Together, this negative feedback loop maintains a homeostatic production of androgens. In pigs, maintaining normal estrogen level is critical important for testicular development. A reduction in estrogen increases testis size by increasing Sertoli cell numbers (38) and treatment with excessive estrogen decreases testis size and Sertoli cell number (39-41). Thus, the balance of androgens and estrogens in both a local testicular environment and within the hypothalamus-pituitary-gonad feedback loop is needed for normal development and function of the gonads in the male pig.

Boar taint and aggressive behaviors are caused by the androgens in the pigs. Replacing the current practice of pig castration with a better (less painful for animals and less costly for producers) is a significant challenge that the swine industry faces. The proposed technology, iNeuter (chemically prevent and/or inhibit boar taint and aggression with EB or form thereof, as discussed herein), can resolve these problems by replacing surgical castration with a single injection within days after birth. When the iNeuter is implanted in the newborn male piglet, it inhibits the development of testis and androgen production. Thus, iNeuter addresses the primary issues facing key stakeholders across the swine industry regarding the issue of surgical castrations.

iNeuter can prevent or inhibit androgen production in pigs, eliminating the necessity of surgical castration, which resolves the animal welfare concerns and employee morale. iNeuter requires only one neonatal injection, while Improvest® requires two injections in adolescent hogs. Furthermore, as market adoption failure of the solution such as Improvest® has proven, cost-effectiveness and easy implementation are paramount for a product to be successful and adopted in the swine industry. Based on the laboratory and field research, the solution provided herein can achieve all of the benefits of surgical castration with none of the drawbacks, and most importantly for the swine industry, would be cost-effective and easy to implement.

A method of chemically inhibiting testis development and inhibiting testosterone is provided herein. After several years of research and testing, iNeuter, a tiny silicone capsule (2 mm in diameter×8 mm in length) that contains a small amount of E2-benzoate (EB) was developed. When implanted subcutaneously in neonatal animals, the capsule is slowly emptied/slow release of EB over a few weeks. In the body, EB is converted into E2, a naturally synthesized form of estrogen, elevating E2 concentration in the blood (42), which during the pre-pubertal period permanently inhibits the expression of KISS1, resulting in the loss of germ cells and Leydig cells (cells producing T). The decrease in androgen synthesis inhibits further development and function of testes and the male reproductive tract.

### Materials/Methods

Proof-of-concept was tested by implanting iNeuter in pre-pubertal laboratory rats. It decreased testis size, and lowered T levels to near female levels, virtually eliminating all mating and aggressive behavior. Thus, iNeuter has the potential to provide all the benefits of surgical castration, without the trauma of cutting and subsequent pain. Further, there were no undesirable side effects, indicating its safety.

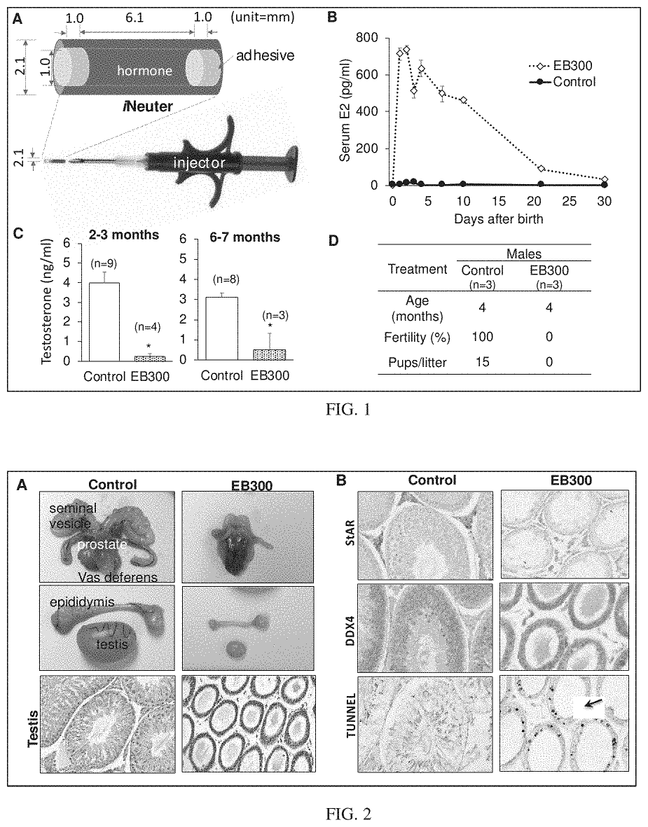

Several iNeuters were made of silicone capsules containing different amounts of EB solutions in corn oil. The silicone capsule was sealed with a medical adhesive on both ends (FIG. 1A). EB capsules containing 30, 100 and 300 μg/pup were implanted subcutaneously in rat pups on the day of birth or 1 day after birth. For simplicity, data from controls (no capsule) and the 300 μg/pup (EB300) group are shown. After implanting EB300, serum concentrations of E2 were measured for up to one month. Serum E2 concentration was elevated and maintained at high levels for 10 days and then gradually decreased to near control levels by day 30 (FIG. 1B). At 2-3 and 6-7 (full sexual maturity) months of age, their blood (serum) levels of sex steroids were measured and fertility was tested. Males had significantly lower T levels (FIG. 1C). Indeed, none of the EB300 implanted males produced litters when they were housed with proven females (FIG. 1D). The rats were euthanized, and their reproductive organs examined. EB300 implanted males had under-developed reproductive organs. Seminal vesicle, testis, and epididymis were remarkably smaller than control (FIG. 2A). Testis histology revealed either no sign of spermatogenesis or in some minimal spermatogenesis, as the seminiferous tubules were mostly devoid of germ cells (FIG. 2A). Staining the testis with StAR (steroidogenesis marker), DDX4 (germ cell marker) and TUNNEL (Apoptosis marker), showed that steroidogenic activity was low, germ cells were depleted, and cell death was elevated in the EB300 implanted males. These results are consistent with significantly low serum T levels and loss of fertility in the implanted males. Furthermore, metabolic analysis, hematology and major organ histology showed no pathological responses in the EB-implanted rats (data not shown). Data from 10-week old pigs show that the testicles in iNeutered pigs are smaller than intact pigs and serum T is greatly reduced.

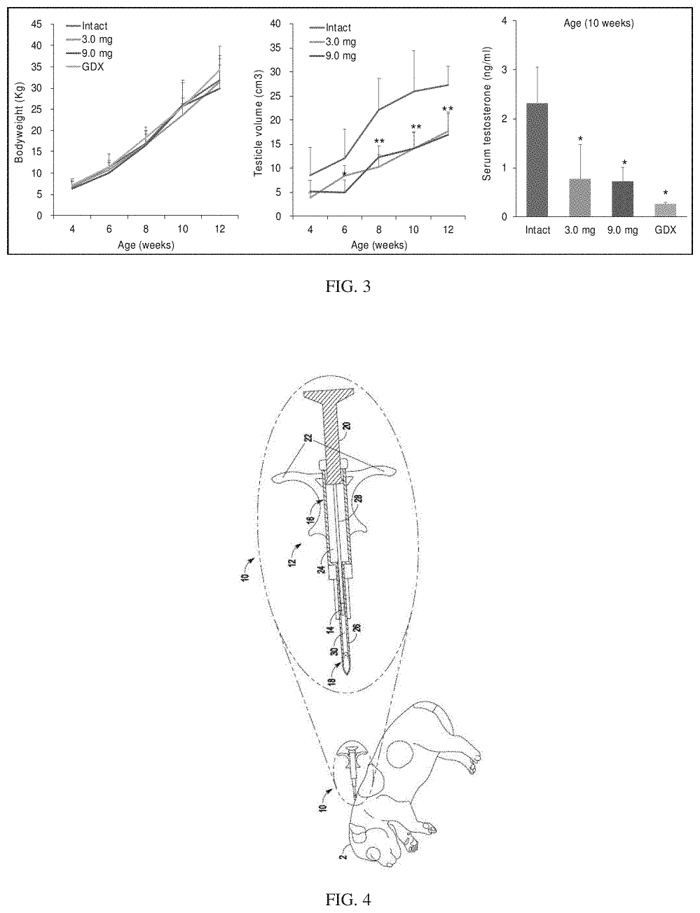

iNeuters were then implanted in piglets and their testicular development and serum testosterone levels measured. The pigs were divided by 4 groups: Intact (control; no implant received), 3.0 mg EB, 9.0 mg EB, and GDX (castrated). The pigs of 3.0 mg group were implanted with 3.0 mg of EB a day after birth (Postnatal day 1 or PND1). The pigs of the 9.0 mg group were implanted with 9.0 mg of EB) on PND1. Pigs of GDX (gonadectomized) group were surgically castrated on PND 1. Sample sizes are n=5 for intact group, n=9 for 3.0 mg group, n=5 for 9.0 mg group and n=5 for GDX group. Presented in FIG. 3 are the patterns of body weight growth (Left; FIG. 3) and testicular volume change (Middle; FIG. 3) during the period of 4 weeks of ages to 12 weeks of ages. Also presented are the serum testosterone concentration measured at the age of 10 weeks (Right; FIG. 3). Note that no statistical difference was seen in the body weight at any time point of measurement and that from the age of 8 weeks the testicular sizes of both 3.0 and 9.0 mg groups were smaller than those of Intact group (p<0.05). At week 10, serum testosterone concentrations of 3.0 and 9.0 mg groups were significantly lower than those of controls (p<0.05), but not different from those of GDX group.

### BIBLIOGRAPHY

1. USDA. Livestock and Poultry: World Markets and Trade 2019 (Available from: fas.usda.gov/data/livestock-and-poultry-world-markets-and-trade).
2. Morales J, et al. Surgical castration with pain relief affects the health and productive performance of pigs in the suckling period. Porcine Health Manag. 2017; 3:18.
3. Telles F G, Luna S P L, Teixeira G, Berto D A. Long-term weight gain and economic impact in pigs castrated under local anaesthesia. Veterinary and Animal Science. 2016; 1-2:36-9.
4. Rault J-L, Lay D, Marchant-Forde J. Castration induced pain in pigs and other livestock. Applied Animal Behaviour Science—APPL ANIM BEHAV SCI. 2011; 135.
5. Rault J-L, Lay Jr D C, Marchant-Forde J N. Farm Animal Welfare Fact Sheet. age. 84:1271-8.
6. American Veterinary Medical Association. Swine Castration 2019 (updated 2019 Oct. 18. Available from: avma.org/KB/Policies/Pages/Swine-Castration.aspx).
7. USDA APHIS. Animal welfare and pig production systems 2017 (Available from: aphis.usda.gov/animal_health/downloads/oie/2017/feb/terrestrial/cmts/GS86%20TAHSC%20Feb2017%20Chap7.X%20AW%20Pig%20Prodxn%20USACmts.pdt).
8. De Briyne N, Berg C, Blaha T, Temple D. Pig castration: will the EU manage to ban pig castration by 2018? Porcine Health Manag. 2016; 2:29.
9. Gottardo F, et al. Pain alleviation during castration of piglets: a comparative study of different farm options. J Anim Sci. 2016; 94(12):5077-88.
10. Di Pasquale J, et al. Towards the Abandonment of Surgical Castration in Pigs: How is Immunocastration Perceived by Italian Consumers? Animals (Basel). 2019; 9(5).
11. Rault J L, Lay D C, Jr., Marchant-Forde J. Farm Animal Welfare Fact Sheet. In: Unit LBR, editor. West Lafayette, Ind.: USDA-ARS-MWA; 2011. p. 1-3.
12. Ison S H, Clutton R E, Di Giminiani P, Rutherford K M. A Review of Pain Assessment in Pigs. Front Vet Sci. 2016; 3:108.
13. Viscardi A V, Turner P V. Use of Meloxicam or Ketoprofen for Piglet Pain Control Following Surgical Castration. Front Vet Sci. 2018; 5:299.
14. Ramos A K, Fuentes A, Carvajal-Suarez M. Self-Reported Occupational Injuries and Perceived Occupational Health Problems among Latino Immigrant Swine Confinement Workers in Missouri. J Environ Public Health. 2018; 2018:8710901.
15. Dunshea F R, et al. Vaccination of boars with a GnRH vaccine (Improvac) eliminates boar taint and increases growth performance. J Anim Sci. 2001; 79(10):2524-35.
16. Brunius C, et al. Early immunocastration of male pigs with Improvac((R))-effect on boar taint, hormones and reproductive organs. Vaccine. 2011; 29(51):9514-20.
17. Poulsen Nautrup B, Van Viaenderen I, Aldaz A, Mah C K. The effect of immunization against gonadotropin-releasing factor on growth performance, carcass characteristics and boar taint relevant to pig producers and the pork packing industry: A meta-analysis. Res Vet Sci. 2018; 119:182-95.
18. Hendrix Genetics. The Alliance to End Surgical Castration of Swine Announces Precision Breeding Successes 2019 (cited 2019 Oct. 18). Available from: https://www.hendrix-genetics.com/en/news/alliance-end-surgical-castration-swine-announces-precision-breeding-successes/.
19. Sonstegard T, Fahrenkrug S, Carlson D. 307 Precision animal breeding to make genetically castrated animals for improved animal welfare and alternative breeding applications. Journal of Animal Science. 2017; 95:149.
20. Candek-Potokar M, Batorek-Lukac N. Alternatives to surgical castration of pigs. Životnov Dni Nauki 2015; 52(5):41-51.
21. Patterson R L S. 5α-androst-16-ene-3-one: Compound responsible for taint in boar fat. Journal of the Science of Food and Agriculture. 1968; 19(1):31-8.
22. Williamson D E, Patterson R L S. A selective immunization procedure against 5α-androstenone in boars. Animal Science. 1982; 35(3):353-60.
23. Lopez-Bote C, Ventanas J. The reduction of boar taint in male pigs by neonatal testosterone administration. Meat Sci. 1988; 22(3):163-71.
24. Claus R, Hoffmann B. Oestrogens, compared to other steroids of testicular origin, in blood plasma of boars. Acta Endocrinol (Copenh). 1980; 94(3):404-11.
25. Babol J, Squires E J, Lundstrom K. Relationship between metabolism of androstenone and skatole in intact male pigs. J Anim Sci. 1999; 77(1):84-92.
26. Griffiths N M, Patterson R L. Human olfactory responses to 5-alpha-androst-16-en-3-one-principal component of boar taint. J Sci Food Agric. 1970; 21(1):4-6.
27. Bonneau M, et al. An international study on the importance of androstenone and skatole for boar taint: I. Presentation of the programme and measurement of boar taint compounds with different analytical procedures. Meat Sci. 2000; 54(3):251-9.
28. Xue J L, et al. Influence of a gonadotropin-releasing hormone agonist on circulating concentrations of luteinizing hormone and testosterone and tissue concentrations of compounds associated with boar taint. J Anim Sci. 1994; 72(5): 1290-8.
29. Daniel M J, Shenoy E V, Box P G. Immunization of pigs against the boar taint steroid androstenone. J Comp Pathol. 1984; 94(2):319-21.
30. Bonneau M. Dufour R, Chouvet C, Roulet C, Meadus W, Squires E J. The effects of immunization against luteinizing hormone-releasing hormone on performance, sexual development, and levels of boar taint-related compounds in intact male pigs. J Anim Sci. 1994; 72(1):14-20.
31. Meloen R H, et al. Efficient immunocastration of male piglets by immunoneutralization of GnRH using a new GnRH-like peptide. Vaccine. 1994; 12(8):741-6.
32. Candek-Potokar M, Skrlep M, Zamaratskaia. G. Immunocastration as Alternative to Surgical Castrationin Pigs. Theriogenology. 2017; 6:109-26.
33. Han X, et al. Mechanistic insight into the role of immunocastration on eliminating skatole in boars. Theriogenology. 2019; 131:32-40.
34. Wang C, et al. Improvac induces immunocastration by affecting testosterone levels and disrupting spermatogenesis in male broiler chickens. Poult Sci. 2019; 98(10:6034-45.
35. Kotani M, et al. The metastasis suppressor gene KiSS-1 encodes kisspeptins, the natural ligands of the orphan G protein-coupled receptor GPR54. J Biol Chem. 2001; 276(37):34631-6.
36. Smith J T, Roseweir A, Millar M, Clarke L I, Millar R P. Stimulation of growth hormone by kisspeptin antagonists in ewes. J Endocrinol. 2018; 237(2): 165-73.
37. Griswold M D. Spermatogenesis: The Commitment to Meiosis. Physiol Rev. 2016; 96(1):1-17.
38. Berger T, McCarthy M, Pearl C A, At-Taras E, Roser J F, Conley A. Reducing endogenous estrogens during the neonatal and juvenile periods affects reproductive tract development and sperm production in postpuberal boars. Anim Reprod Sci. 2008; 109(1-4):218-35.
39. Berger T, Conley A. Reduced Endogenous Estrogen and Hemicastration Interact Synergistically to Increase Porcine Sertoli Cell Proliferation1. Biology of Reproduction. 2014; 90(5).
40. Berger T, Nitta-Oda B J. Increased testicular estradiol during the neonatal interval reduces Sertoli cell numbers. Anim Reprod Sci. 2018.
41. Berger T, Conley A J, Van Klompenberg M, Roser J F, Hovey R C. Increased testicular Sertoli cell population induced by an estrogen receptor antagonist. Mol Cell Endocrinol. 2013; 366(1):53-8.
42. Eaton G G, Goy R W, Resko J A. Brain uptake and metabolism of estradiol benzoate and estrous behavior in ovariectomized guinea pigs. Hormones and Behavior. 1975; 6(1):81-97.
43. Missikpode C, Peek-Asa C, Young T, Swanton A, Leinenkugel K, Torner J. Trends in non-fatal agricultural injuries requiring trauma care. Injury epidemiology. 2015; 2(1):30.
44. Dvorak M. The development of piglets after neonatal venipuncture or orchidectomy. Veterinarni medicina. 1980; 25(1):11-9.
45. National Pork Producers Council. Pork Facts 2019 (cited 2019 Oct. 18) available from: nppc.org/pork-facts/.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof) or with respect to other examples (or one or more aspects thereof) shown or described herein.

In the event of inconsistent usages between this document and any documents so incorporated by reference, the usage in this document controls.

The above description is intended to be illustrative and not restrictive. For example, the above-described examples (or one or more aspects thereof) may be used in combination with each other. Other embodiments can be used, such as “by one of ordinary skill in the art” upon reviewing the above description. The Abstract is provided to comply with 37 C.F.R. § 1.72(b) to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in fewer than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims along with the full scope of equivalents to which such claims are entitled.

What is claimed is:

1. A method to prevent or inhibit boar taint and/or aggression comprising administering to said pig an effective amount of estradiol, estradiol benzoate, estradiol dipropionate, estradiol valerate, estradiol cypionate or a combination thereof so as to prevent or inhibit boar taint and/or aggression.

2. The method of claim 1, wherein the estradiol, estradiol benzoate, estradiol dipropionate, estradiol valerate, estradiol cypionate, or a combination thereof is administered in a composition comprising a carrier.

3. The method of claim 2, wherein the carrier is a physiologically acceptable oil.

4. The method of claim 1, wherein the estradiol, estradiol benzoate, estradiol dipropionate, estradiol valerate, estradiol cypionate, or a combination thereof is formulated in a slow release formulation.

5. The method of claim 1, wherein the administration is a single dose, one time, administration.

6. The method of claim 1, wherein the estradiol, estradiol benzoate, estradiol dipropionate, estradiol valerate, estradiol cypionate, or a combination thereof is administered intraperitoneally, intramuscularly, or subcutaneously.

7. The method of claim 1, wherein the estradiol, estradiol benzoate, estradiol dipropionate, estradiol valerate, estradiol cypionate, or a combination thereof is administered prior to puberty.

8. The method of claim 1, wherein the estradiol, estradiol benzoate, estradiol dipropionate, estradiol valerate, estradiol cypionate, or a combination thereof is administered top the male pig within 1-7 days after birth.

9. The method of claim 1, wherein the estradiol, estradiol benzoate, estradiol dipropionate, estradiol valerate, estradiol cypionate, or a combination thereof is administered to the male pig within 3 days after birth.

10. The method of claim 1, wherein between about 0.3 mg to about 11.0 mg estradiol, estradiol benzoate, estradiol dipropionate, estradiol valerate, estradiol cypionate, or a combination thereof is administered to the male pig.

11. The method of claim 1, wherein about 3 mg or about 9.0 mg estradiol, estradiol benzoate, estradiol dipropionate, estradiol valerate, estradiol cypionate, or a combination thereof is administered to the male pig.
